(19) United States
(12) Patent Application Publication
(10) Pub. No.: US 2019/0159860 A1
(43) Pub. Date: May 30, 2019

(54) PHOTOGRAPHING SYSTEM, PHOTOGRAPHING METHOD AND CONTROL DEVICE

(71) Applicant: SHARP KABUSHIKI KAISHA, Sakai City, Osaka (JP)

(72) Inventor: OSAMU TERANUMA, Sakai City (JP)

(73) Assignee: SHARP KABUSHIKI KAISHA, Sakai City, Osaka (JP)

(21) Appl. No.: 16/321,913

(22) PCT Filed: May 31, 2017
(86) PCT No.: PCT/JP2017/020224

§ 371 Date: Jan. 30, 2019

(30) Foreign Application Priority Data
Aug. 3, 2016 (JP) 2016-152503

(51) Int. Cl. A61B 90/00; A61B 1/05; A61B 1/00; A61B 1/313

(52) U.S. Cl. CPC A61B 90/361; A61B 1/3132; A61B 1/00045; A61B 1/05

(57) ABSTRACT

There is provided an image photographing system including a camera and a control device configured to enable communication. The control device adjusts an image photographing direction of the camera based on an orientation of the camera and an orientation of a prescribed subject. Further, provided is an image photographing method including a step of acquiring an orientation of the camera, a step of acquiring an orientation of the prescribed subject, and a step of adjusting an image photographing direction of the camera based on the orientation of the camera and the orientation of the prescribed subject.

### TECHNICAL FIELD

This application claims the benefit of priority to JP 2016-152503, filed on Aug. 3, 2016. The entire contents of the above-identified application are hereby incorporated by reference.

The following disclosure relates to technologies of image capturing systems, image capturing methods and control devices that can be used in laparoscopic surgery and the like.

### BACKGROUND

Conventionally, technologies related to image capturing systems, image capturing methods and control devices that can be used in laparoscopic surgery and the like have been widely known. For example, JP 4860629 B (PTL 1) discloses a laparoscopic surgery monitor device and a display method for the stated monitor device. According to PTL 1, the laparoscopic surgery monitor device includes: a plurality of laparoscope monitors of flat panel type on which picture screens captured by a single laparoscope unit are respectively applied and displayed, and each of which is supported from the ceiling or the floor with an arm in such a manner that a position, a height, and rightward and leftward slant angles of the laparoscope monitor are adjustable; screen rotation operation units provided for each of the laparoscope monitors; drive motors attached to rear surfaces of the respective laparoscope monitors for rotating the laparoscope monitors in the clockwise direction and in the counterclockwise direction; motor drivers for rotationally driving the drive motors; a control unit configured to rotate each laparoscope monitor, in response to a request for rotation from a practitioner of surgery through the screen rotation operation unit, by the requested amount of rotation angle by controlling the driving of the motor driver based on the request for rotation from the screen rotation operation unit; and protection boxes, each of which is supported from a leading end of the arm in a non-rotatable manner with respect to the leading end, accommodates the laparoscope monitor supported by the arm, has a circular opening window in a front surface thereof, and covers the image screen of the laparoscope monitor around the opening window.

### CITATION LIST

### Patent Literature

PTL 1: JP 4860629 B

### SUMMARY OF DISCLOSURE

### Technical Problem

An object of an aspect of the present disclosure is to provide an image capturing system, an image capturing method, and a control device that can be used by medical specialists such as an operating surgeon more easily than those of the prior art.

### Solution to Problem

According to a certain aspect of the present disclosure, an image capturing system including a camera, and a control device configured to communicate with the camera is provided. The control device is configured to adjust an image capturing direction of the camera based on the orientation of the camera and the orientation of the prescribed subject.

It is preferable for the image capturing system to include a display. The control device causes an image captured by the camera to be rotated based on an orientation of the camera and an orientation of a prescribed subject, and then outputs the image on the display.

It is preferable for the control device to accept designation of the prescribed subject for each user.

According to still another aspect of the present disclosure, provided is an image capturing method including a step of acquiring an orientation of a camera, a step of acquiring an orientation of a prescribed subject, and a step of adjusting the image capturing direction of the camera based on the orientation of the camera and the orientation of the prescribed subject.

According to another aspect of the present disclosure, there is provided a control device including a communication interface configured to communicate with a camera and a processor. The processor adjusts an image capturing direction of the camera based on an orientation of the camera and an orientation of the prescribed subject through the communication interface.

### Advantageous Effects of Disclosure

As discussed above, according to an aspect of the present disclosure, there are provided an image capturing system, an image capturing method and a control device that can be used by medical specialists more easily than those of the prior art.

### BRIEF DESCRIPTION OF DRAWINGS

FIG. 1 is a first image diagram illustrating an overall configuration and an operational outline of an image output system 1 according to a first embodiment.

FIG. 2 is a second image diagram illustrating the overall configuration and the operational outline of the image output system 1 according to the first embodiment.

FIG. 3 is a block diagram illustrating a hardware configuration of the image output system 1 according to the first embodiment.

FIG. 4 is a block diagram illustrating a hardware configuration of a control device 100 according to the first embodiment.

FIG. 5 is an image diagram illustrating a structure of a camera 200A according to the first embodiment.

FIG. 6 is a flowchart illustrating a first information process in the control device 100 according to the first embodiment.

FIG. 7 is a first image diagram illustrating an overall configuration and an operational outline of an image output system 1 according to a second embodiment.

FIG. 8 is a second image diagram illustrating an overall configuration and an operational outline of the image output system 1 according to the second embodiment.

FIG. 9 is a flowchart illustrating a first information process in a control device 100 according to the second embodiment.

FIG. 10 is a first image diagram illustrating an overall configuration and an operational outline of an image output system 1 according to a third embodiment.

FIG. 11 is a second image diagram illustrating the overall configuration and the operational outline of the image output system 1 according to the third embodiment.

FIG. 12 is a flowchart illustrating a first information process in a control device 100 according to the third embodiment.

FIG. 13 is a first image diagram illustrating an overall configuration and an operational outline of an image output system 1 according to a fourth embodiment.

FIG. 14 is a second image diagram illustrating the overall configuration and the operational outline of the image output system 1 according to the fourth embodiment.

FIG. 15 is a flowchart illustrating a first information process in a control device 100 according to the fourth embodiment.

FIG. 16 is a flowchart illustrating a first information process in a control device 100 according to a fifth embodiment.

FIG. 17 is a plan view illustrating a relationship in orientation and a positional relationship among a camera 200A, the body of an operating surgeon, the head of the operating surgeon and a display 300, after changing an image photographing direction of the camera 200A in accordance with an orientation of a subject according to the fifth embodiment.

FIG. 18 is an image diagram illustrating a relationship among an image photographing direction of the camera 200A, an orientation of the body of the operating surgeon, a line-of-sight direction of the operating surgeon, and an orientation of the display 300, after changing an image photographing direction of the camera 200A in accordance with the orientation of the subject according to the fifth embodiment.

FIG. 19 is a first flowchart illustrating a second information process in a control device 100 according to the fifth embodiment.

FIG. 20 is a second flowchart illustrating the second information process in the control device 100 according to the fifth embodiment.

FIG. 21 is an image diagram illustrating actual operations of an image output system 1 according to the fifth embodiment.

FIG. 22 is an image diagram illustrating actual operation results in the display 300 of the image output system 1 according to the fifth embodiment.

FIG. 23 is a flowchart illustrating a first information process in a control device 100 according to a sixth embodiment.

### DESCRIPTION OF EMBODIMENTS

Hereinafter, embodiments of the present disclosure will be described with reference to the accompanying drawings. Note that in the following description, identical constituent elements are assigned the same reference signs. The above-mentioned constituent elements have the identical names and identical functions as well. Accordingly, detailed description thereof will not be repeated.

### First Embodiment

### Overall Configuration and Operational Outline of Picture Output System

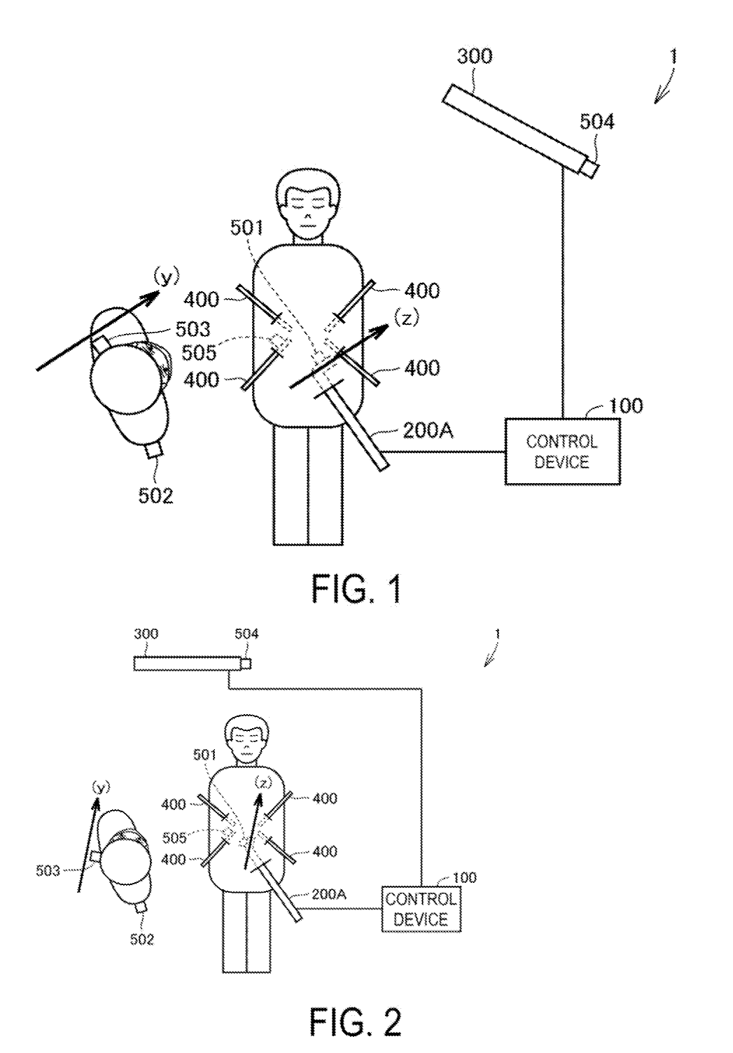

Hereinafter, an image output system including a display, among an image photographing system, will be described. First, with reference to FIGS. 1 and 2, an overall configuration and an operational outline of an image output system 1 according to the present embodiment will be described. FIG. 1 is a first image diagram illustrating an overall configuration and an operational outline of the image output system 1 according to the present embodiment. FIG. 2 is a second image diagram illustrating the overall configuration and the operational outline of the image output system 1 according to the present embodiment.

First, the overall configuration of the image output system 1 according to the present embodiment will be described. The image output system 1 according to the present embodiment primarily includes a camera 200A such as a laparoscope, a display 300, and a control device 100 configured to control the camera 200A and the display 300. The operational outline of the image output system 1 according to the present embodiment will be described below.

The control device 100 causes an image photographed by the camera 200A to be displayed on the display 300. In particular, the control device 100 changes the image photographing direction of the camera 200A and displays the photographed image on the display 300 so that the photographed image can be easily seen by the operating surgeon as a medical specialist, a positional relationship among the organs being displayed, treatment instruments and the like can be easily recognized by the operating surgeon, and the operation can be easily performed by the operating surgeon.

For example, as illustrated in FIG. 1, in a case where the operating surgeon is looking at the display 300, placed at an upper right side of a patient, from the left side of the patient when seen from above, a tip end of the camera 200A changes the orientation toward a direction (z) that is similar to a line-of-sight direction (y) of the operating surgeon, that is the upper right direction in FIG. 1, for photographing images of the inside of the patient. Then, as illustrated in FIG. 2, in a case where the operating surgeon is looking at the display 300, placed at an upper side of a patient, from the left side of the patient when seen from above, the tip end of the camera 200A changes the orientation toward a direction (z) similar to the line-of-sight direction (y) of the operating surgeon, that is the upper direction in FIG. 1, for photographing images of the inside of the patient.

Hereinafter, a specific configuration of the image output system 1 for enabling the above-mentioned functions will be described in detail.

### Hardware Configuration of Picture Output System 1

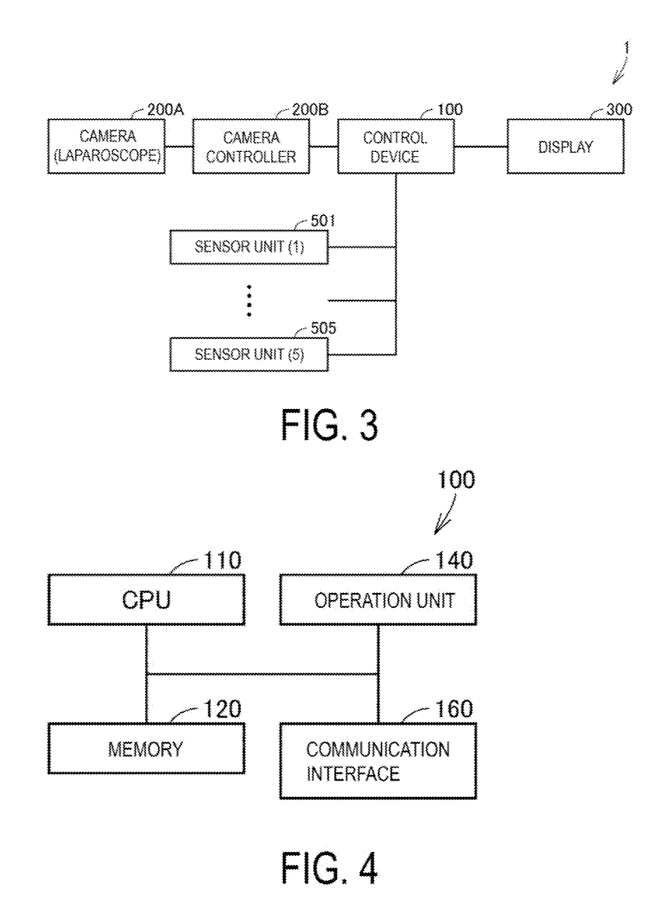

First, an aspect of the hardware configuration of the image output system 1 according to the present embodiment will be described. FIG. 3 is a block diagram illustrating the hardware configuration of the image output system 1 according to the present embodiment.

Referring to FIG. 3, the image output system 1 according to the present embodiment includes the camera 200A for photographing a treatment site, an examination site or the like, a camera controller 200B configured to control the camera 200A, the display 300 to which an image of the treatment site, the examination site or the like is outputted, the control device 100 configured to control the above-mentioned devices, sensor units 501, 502, 503, 504 and 505 for measuring positions, postures and the like of the operating surgeon, the patient and the above devices, and the like.

The first sensor unit 501 according to the present embodiment is attached to the camera 200A, and reports, to the control device 100, an image photographing direction of the camera 200A, a posture of the camera 200A, an angle indicating a slant of the photographed image of the camera 200A relative to a vertical upper side, and the like, by making use of an electronic compass or a magnet installed inside the sensor unit 501. The first sensor unit 501 may also acquire a position of the camera 200A and may transmit the position of the camera 200A to the control device 100. The first sensor unit 501 may be included in the camera 200A or may be integrated with the camera 200A.

The second sensor unit 502 according to the present embodiment is attached to the body or the clothes of the operating surgeon, and reports, to the control device 100, the orientation of the body of the operating surgeon by making use of an electronic compass or a magnet installed inside the sensor unit 502. The second sensor unit 502 may also acquire a position of the body of the operating surgeon and may transmit the position of the body of the operating surgeon to the control device 100.

The third sensor unit 503 according to the present embodiment is mounted on head of the operating surgeon, and reports, to the control device 100, a line-of-sight direction of the operating surgeon or an orientation of the face of the operating surgeon by making use of an electronic compass or a magnet installed inside the sensor unit 503. The third sensor unit 503 may also acquire a position of the head of the operating surgeon and may transmit the position of the head of the operating surgeon to the control device 100.

A fourth sensor unit 504 according to the present embodiment is attached to the display 300, and reports, to the control device 100, the orientation of the display 300 by making use of an electronic compass or a magnet installed inside the sensor unit 504. The fourth sensor unit 504 may also acquire a position of the display 300 and may transmit the position of the display 300 to the control device 100. In addition, the fourth sensor unit 504 may be included in the display 300 or may be integrated with the display 300.

The filth sensor unit 505 according to the present embodiment is attached to the treatment instrument 400, and reports, to the control device 100, the orientation of the treatment instrument 400 by making use of an electronic compass or a magnet installed inside the sensor unit 505. The fifth sensor unit 505 may also acquire a position of the treatment instrument 400 and may transmit the position of the treatment instrument 400 to the control device 100. The fifth sensor unit 505 may be included in the treatment instrument 400 or may be integrated with the treatment instrument 400.

In order for the first to fifth sensor units 501 to 505 to accurately detect the direction, posture, and position of the camera 200A, the direction, posture, and position of the body of the operating surgeon, the direction, posture, and position of the head of the operating surgeon, the direction, posture, and position of the display 300, and the direction, posture, and position of the treatment instrument 400, an indoor GPS antenna, a WiFi (trade name) router, an ultrasonic wave oscillator, or the like may be disposed in four corners or the like of the operating room.

An aspect of the hardware configuration of the control device 100 according to the present embodiment will be described hereinafter. FIG. 4 is a block diagram illustrating the hardware configuration of the control device 100 according to the present embodiment.

Referring to FIG. 4, the control device 100 includes, as main constituent elements, a Central Processing Unit (CPU) 110, a memory 120, an operation unit 140 and a communication interface 160.

The CPU 110 controls constituent elements of the control device 100 by performing programs stored in the memory 120. To be specific, the CPU 110 carries out various processes to be explained later by performing the programs stored in the memory 120 and referring to various kinds of data.

The memory 120 is implemented by various types of Random Access Memories (RAMs), various types of Read-Only Memories (ROMs), or the like. The memory 120 stores the programs to be performed by the CPU 110, data created by the CPU 110 performing the programs, inputted data, and other data such as a database.

The operation unit 140 accepts commands from a user and inputs the stated commands to the CPU 110. The operation unit 140 may be a touch panel including a display.

The communication interface 160 receives data from devices such as the camera controller 200B and the display 300 to deliver the received data to the CPU 110, transmits data from the CPU 110 to the devices such as the camera controller 200B, the display 300, and the like. Note that the communication interface 160 may exchange data with other external apparatuses such as a server via the Internet, a router or the like.

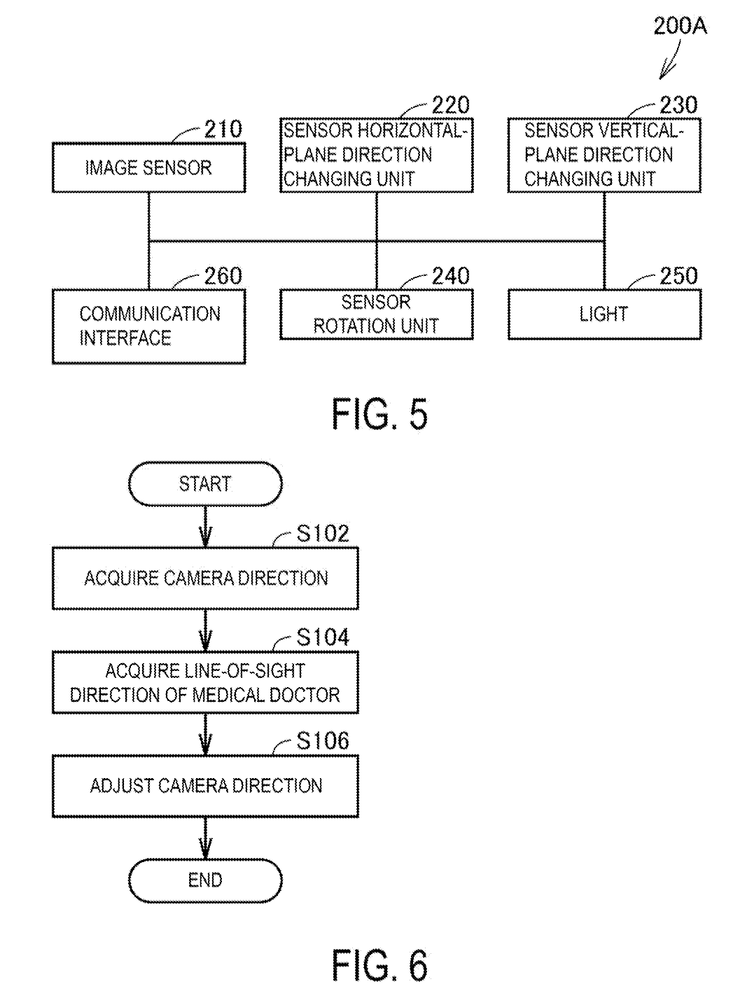

Next, an aspect of the hardware configuration of the camera 200A according to the present embodiment will be described. FIG. 5 is an image diagram illustrating a structure of the camera 200A according to the present embodiment. In the present embodiment, the camera 200A primarily includes an image sensor 210, a sensor horizontal direction changing unit 220, a sensor vertical direction changing unit 230, a sensor rotation unit 240, a light 250, and a communication interface 260.

The image sensor 210 detects light and outputs a signal to represent an image. To be more specific, the camera 200A radiates light from the light 250, and reflection light thereof is detected by the image sensor 210.

The communication interface 260 transmits images photographed by the image sensor 210 to a camera controller 200B, a control device 100 and the like.

The sensor horizontal direction changing unit 220 is constituted of a motor, an actuator, and the like, and changes a direction of a horizontal component photographed by the image sensor 210, based on a command from the control device 100 inputted through the communication interface 260.

The sensor vertical direction changing unit 230 is constituted of a motor, an actuator, and the like, and changes a direction of a vertical component photographed by the image sensor 210, based on a command from the control device 100 inputted through the communication interface 260.

The sensor rotation unit 240 is constituted of a motor, an actuator, and the like, and makes the image sensor 210 rotate while taking an image photographing direction of the image sensor 210 as an axis, based on a command from the control device 100 inputted through the communication interface 260.

### Information Process in Control Device 100

Next, a first information process in a control device 100 according the present embodiment will be described. FIG. 6 is a flowchart illustrating the first information process in the control device 100 according to the present embodiment.

Referring to FIG. 6, a CPU 110 of the control device 100 periodically acquires, from a first sensor unit 501 through a communication interface 160, vector data indicating an image photographing direction (z) of the camera 200A (step S102).

The CPU 110 acquires, from the third sensor unit 503 through the communication interface 160, vector data indicating a line-of-sight direction (y) of the operating surgeon (step S104).

The CPU 110 causes the image photographing direction (z) of the camera 200A to turn toward the identical direction with the line-of-sight direction (y) of the operating surgeon (step S106). To be more specific, the CPU 110 transmits, to the camera 200A through the communication interface 160, a command to control a sensor horizontal direction changing unit 220 and a sensor vertical direction changing unit 230 of the camera 200A, and thus the image photographing direction (z) of the camera 200A is made to turn toward the line-of-sight direction (y) of the operating surgeon. Then, the CPU 110 transmits, to the camera 200A through the communication interface 160, a command to control a sensor rotation unit 240 of the camera 200A, and thus an image sensor 210 at the tip end of the camera 200A is rotated taking a photographing direction as an axis when the upper side of the photographed image comes closest to the actual vertical upper side.

Thus, the CPU 110 of the control device 100 acquires image data from the camera 200A through the communication interface 160. The CPU 110 displays pictures photographed from the camera 200A toward the line-of-sight direction of the operating surgeon on the display 300 through the communication interface 160.

The frequency of the changes in image photographing directions of the camera 200A may be adjustable (e.g., 60 seconds, 10 seconds, one second, or timing determined by the operating surgeon by manual operation). Alternatively, a configuration may be selected in which the positional relationship among the devices is “confirmed” before the operation, and the image photographing direction of the camera 200A is not changed during the operation.

### Second Embodiment

In the first embodiment, the direction of the image sensor 210 of the camera 200A is changed based on the line-of-sight direction of the operating surgeon. However, the present disclosure is not limited to such embodiment. In the present embodiment, the direction of the image sensor 210 of the camera 200A is changed based on the orientation of the body of the operating surgeon.

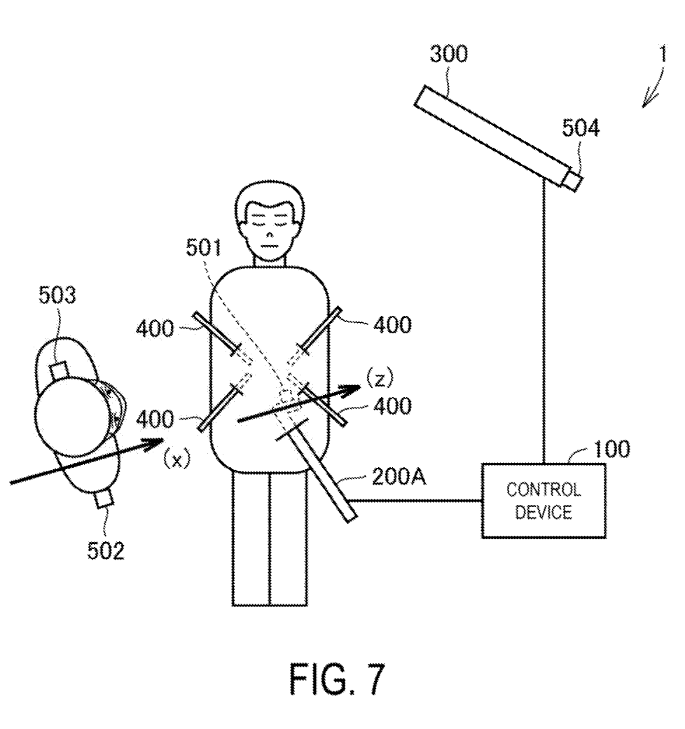

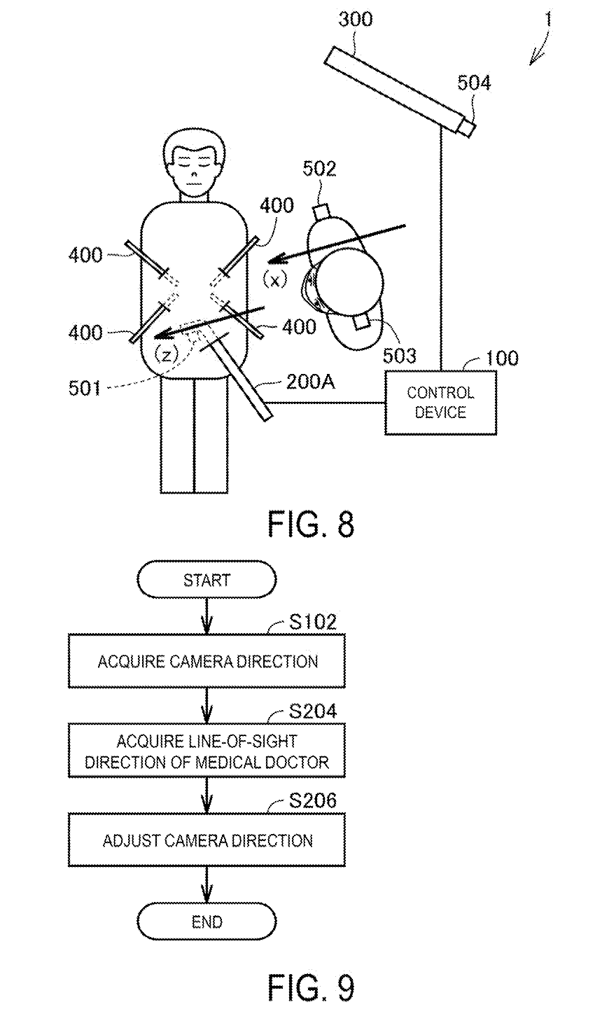

With reference to FIGS. 7 and 8, an operational outline of an image output system 1 according to the present embodiment will be described. FIG. 7 is a first image diagram illustrating an overall configuration and the operational outline of the image output system 1 according to the present embodiment. FIG. 8 is a second image diagram illustrating the overall configuration and the operational outline of the image output system 1 according to the present embodiment.

Since the overall configuration of the image output system 1 according to the present embodiment is similar to that of the first embodiment, description thereof will not be repeated herein. The operational outline of the image output system 1 according to the present embodiment will be described below.

In the present embodiment, as illustrated in FIG. 7, in a case where the body of the operating surgeon is turned toward the direction of the patient from the left side of the patient, a tip end of the camera 200A changes the orientation toward a direction similar to an orientation (x) of the body of the operating surgeon, that is the right direction in FIG. 1, for photographing images of the inside of the patient. Then, as illustrated in FIG. 8, in a case where the operating surgeon is facing the direction of the patient, from the right side of the patient, the tip end of the camera 200A changes the orientation similar to an orientation (x) of the body of the operating surgeon, that is the upper left direction in FIG. 8, for photographing images of the inside of the patient.

Next, a first information process in the control device 100 according to the present embodiment will be described. FIG. 9 is a flowchart illustrating the first information process in the control device 100 according to the present embodiment.

Referring to FIG. 9, a CPU 110 of the control device 100 periodically acquires, from a first sensor unit 501 through a communication interface 160, vector data indicating an image photographing direction (z) of the camera 200A (step S102).

The CPU 110 acquires, from a second sensor unit 502 through the communication interface 160, vector data indicating an orientation (x) of the body of the operating surgeon (step S204).

The CPU 110 causes the image photographing direction (z) of the camera 200A to turn toward the orientation (x) of the body of the operating surgeon (step S206). To be more specific, the CPU 110 transmits, to the camera 200A through the communication interface 160, a command to control a sensor horizontal direction changing unit 220 and a sensor vertical direction changing unit 230 of the camera 200A, and thus the image photographing direction (z) of the camera 200A is made to turn toward the orientation (x) of the body of the operating surgeon. Then, the CPU 110 transmits, to the camera 200A through the communication interface 160, a command to control a sensor rotation unit 240 of the camera 200A, and thus the image sensor 210 at the tip end of the camera 200A is rotated taking the image photographing direction as an axis so that the upper side of the photographed image comes closest to the actual vertical upper side.

Thus, the CPU 110 of the control device 100 acquires image data from the camera 200A through the communication interface 160. The CPU 110 causes the image photographed in the direction of the orientation of the body of the operating surgeon by the camera 200A to be displayed on the display 300 through the communication interface 160.

### Third Embodiment

In the first embodiment, the direction of the image sensor 210 of the camera 200A is changed based on the line-of-sight direction of the operating surgeon. However, the present disclosure is not limited to such embodiment. In the present embodiment, a direction of an image sensor 210 of a camera 200A is changed based on a direction of a treatment instrument 400.

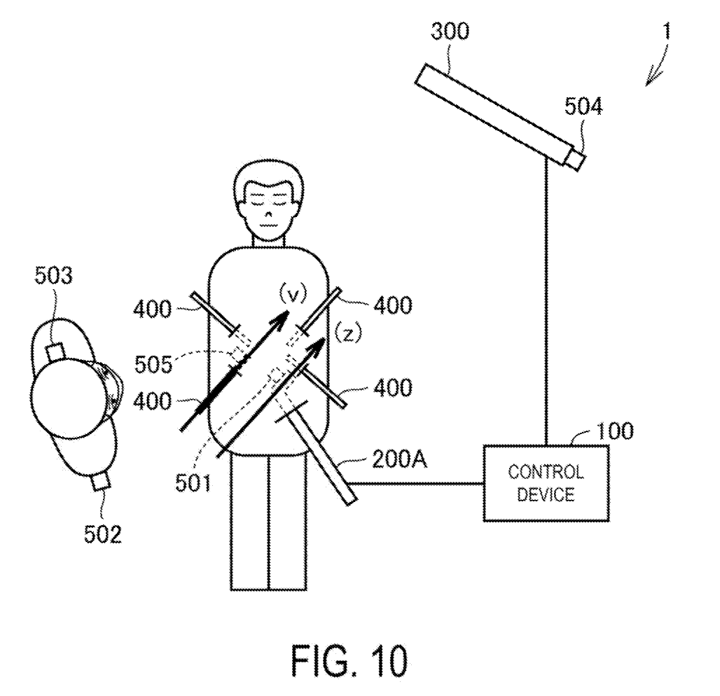

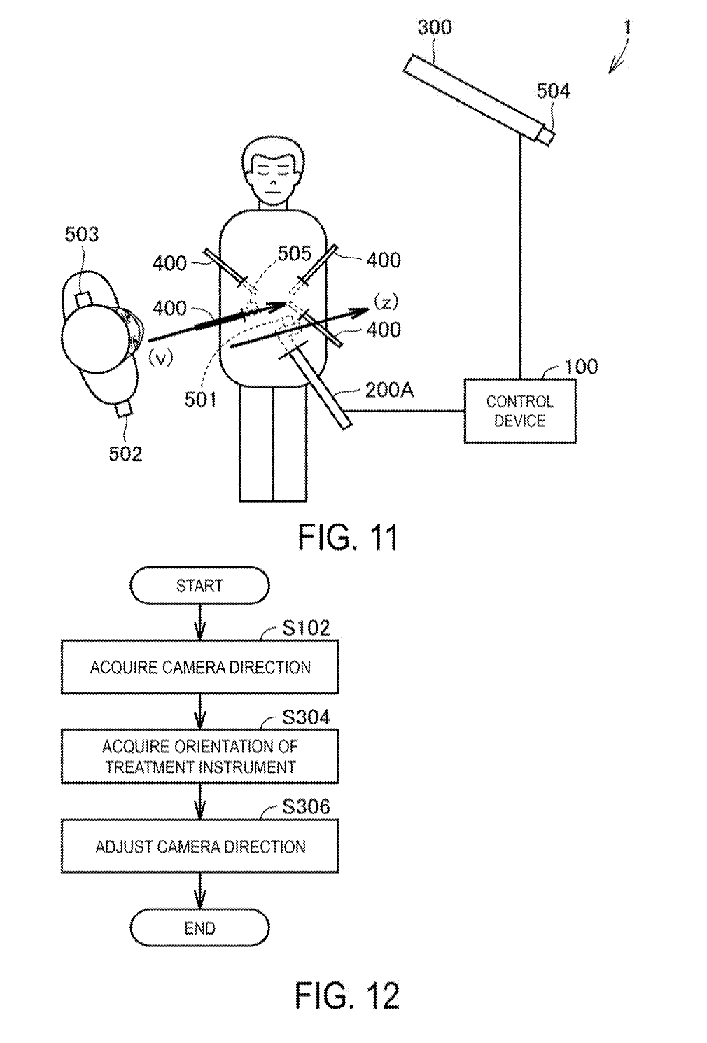

With reference to FIGS. 10 and 11, an operational outline of an image output system 1 according to the present embodiment will be described. FIG. 10 is a first image diagram illustrating an overall configuration and the operational outline of the image output system 1 according to the present embodiment. FIG. 11 is a second image diagram illustrating an overall configuration and an operational outline of the image output system 1 according to the present embodiment.

Since the overall configuration of the image output system 1 according to the present embodiment is similar to that of the first embodiment, description thereof will not be repeated herein. The operational outline of the image output system 1 according to the present embodiment will be described below.

In the present embodiment, as illustrated in FIG. 10, in a case where the treatment instrument 400 being currently used by the operating surgeon is turned toward the upper right side from the lower left side of the patient, a tip end of the camera 200A changes the orientation toward the upper right direction in FIG. 10, for photographing images of the inside of the patient. Then, as illustrated in FIG. 11, in a case where the treatment instrument 400 being currently used by the operating surgeon is turned from the left side toward the right side of the patient, the tip end of the camera 200A changes the orientation toward the right direction in FIG. 11 for photographing images of the inside of the patient.

Next, a first information process in the control device 100 according to the present embodiment will be described. FIG. 12 is a flowchart illustrating a first information process in the control device 100 according to the present embodiment.

Referring to FIG. 12, a CPU 110 of the control device 100 periodically acquires, from a first sensor unit 501 through a communication interface 160, vector data indicating an image photographing direction (z) of the camera 200A (step S102).

The CPU 110 acquires, from a sensor unit 505 being attached to the treatment instrument 400 through the communication interface 160, vector data indicating an orientation (v) of the treatment instrument 400 (step S304).

The CPU 110 causes the image photographing direction of the camera 200A to turn toward the orientation (v) of the treatment instrument 400 (step S306). To be more specific, the CPU 110 transmits, to the camera 200A through the communication interface 160, a command to control a sensor horizontal direction changing unit 220 and a sensor vertical direction changing unit 230 of the camera 200A, and thus the image photographing direction (z) of the camera 200A is made to turn toward the orientation (v) of the treatment instrument 400. Then, the CPU 110 transmits, to the camera 200A through the communication interface 160, a command to control a sensor rotation unit 240 of the camera 200A, and thus the image sensor 210 at the tip end of the camera 200A is rotated taking the image photographing direction as an axis so that the upper side of the photographed image comes closest to the actual vertical upper side.

Thus, the CPU 110 of the control device 100 acquires image data from the camera 200A through the communication interface 160. The CPU 110 causes the image photographed in the identical orientation to that of the treatment instrument 400 by the camera 200A to be displayed on the display 300 through the communication interface 160.

### Fourth Embodiment

In the first embodiment, the direction of the image sensor 210 of the camera 200A is changed based on the line-of-sight direction of the operating surgeon. However, the present disclosure is not limited to such embodiment. In the present embodiment, a direction of an image sensor 210 of a camera 200A is to be changed based on the orientation of a display 300.

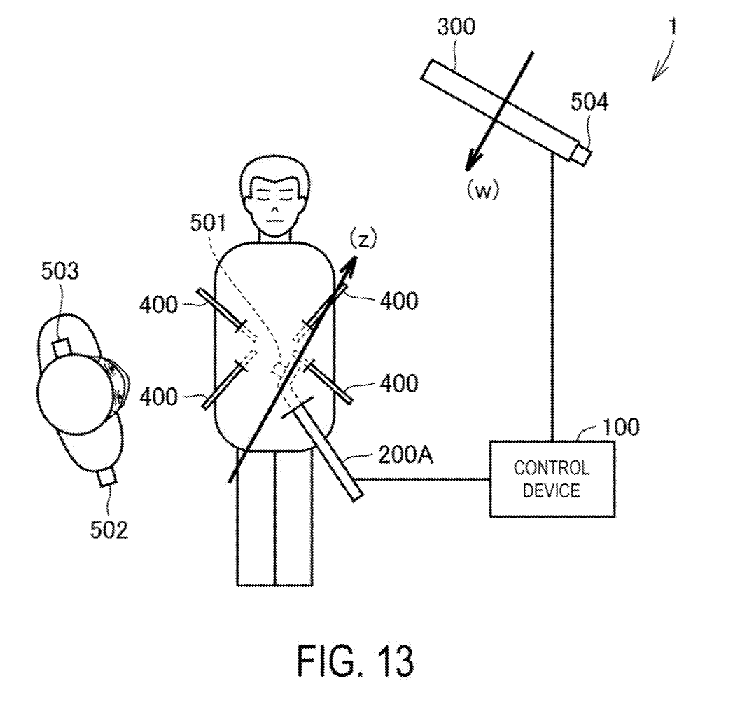

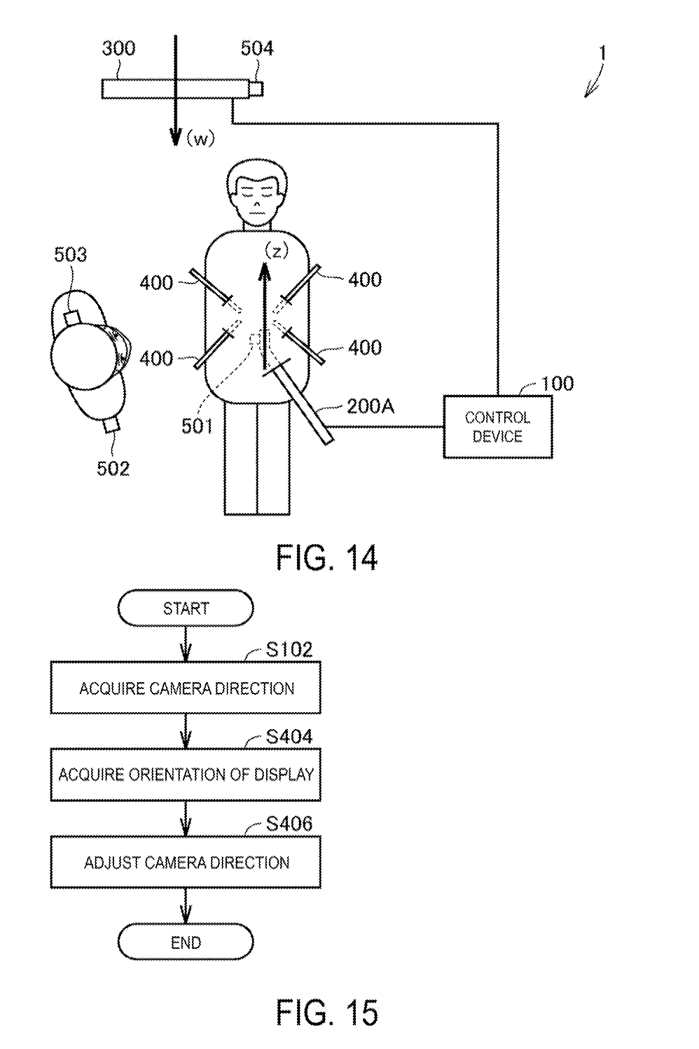

With reference to FIGS. 13 and 14, an operational outline of an image output system 1 according to the present embodiment will he described. FIG. 13 is a first image diagram illustrating an overall configuration and the operational outline of the image output system 1 according to the present embodiment. FIG. 14 is a second image diagram illustrating the overall configuration and the operational outline of the image output system 1 according to the present embodiment.

Since the overall configuration of the image output system 1 according to the present embodiment is similar to that of the first embodiment, description thereof will not be repeated herein. The operational outline of the image output system 1 according to the present embodiment will be described below.

According to the present embodiment, as illustrated in FIG. 13, in a case where the display 300 is turned toward the lower left direction of the display 300, a tip end of the camera 200A changes the orientation toward a direction opposite to the orientation of the display 300, that is the upper right direction in FIG. 13, for photographing images of the inside of the patient. Then, as illustrated in FIG. 14, in a case where the display 300 is turned toward the lower direction, the tip end of the camera 200A changes the orientation toward a direction opposite to the orientation of the display 300, that is the upper direction in FIG. 14, for photographing images of the inside of the patient.

Next, a first information process in a control device 100 according to the present embodiment will be described. FIG. 15 is a flowchart illustrating a first information process in the control device 100 according to the present embodiment.

Referring to FIG. 15, a CPU 110 of the control device 100 periodically acquires, from a first sensor unit 501 through a communication interface 160, vector data indicating an image photographing direction (z) of the camera 200A (step S102).

The CPU 110 acquires, from a sensor unit 504 attached to the display 300 through the communication interface 160, vector data indicating an orientation (w) of the display (step S404).

The CPU 110 causes the image photographing direction (z) of the camera 200A to turn toward the opposite direction to the orientation (w) of the display 300 (step S406). To be more specific, the CPU 110 transmits, to the camera 200A through the communication interface 160, a command to control a sensor horizontal direction changing unit 220 and a sensor vertical direction changing unit 230 of the camera 200A, and thus the image photographing direction (z) of the camera 200A is made to turn toward the orientation opposite to the orientation (w) of the display 300. Then, the CPU 110 transmits, to the camera 200A through the communication interface 160, a command to control a sensor rotation unit 240 of the camera 200A, and thus the image sensor 210 at the tip end of the camera 200A is rotated taking the image photographing direction as an axis so that the upper side of the photographed image comes closest to the actual vertical upper side.

Thus, the CPU 110 of the control device 100 acquires image data from the camera 200A through the communication interface 160. The CPU 110 causes the image photographed in the opposite direction to the orientation of the display 300 by the camera 200A to be displayed on the display 300 through the communication interface 160.

### Fifth Embodiment

In the first to fourth embodiments, the direction of the image sensor 210 of the camera 200A is changed base on the orientation of the subject. However, in some cases, it may be difficult to match the orientation of the image sensor 210 to the orientation of the subject because of the position of the tip end of the camera 200A, the orientation of the subject or the like. Thus, in the present embodiment, an orientation of an image sensor 210 of a camera 200A is made to be as close as possible to an orientation of a subject. While in the case where the orientation of the image sensor 210 of the camera 200A and the orientation of the subject cannot be matched completely, a photographed image is caused to be rotated and displayed, or the image sensor 210 itself is caused to be rotated so as to shift an upper direction of the photographed image of the image sensor 210 from a vertical upper direction.

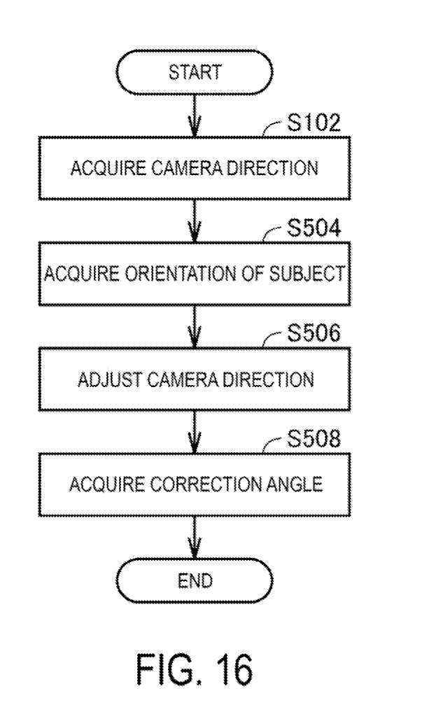

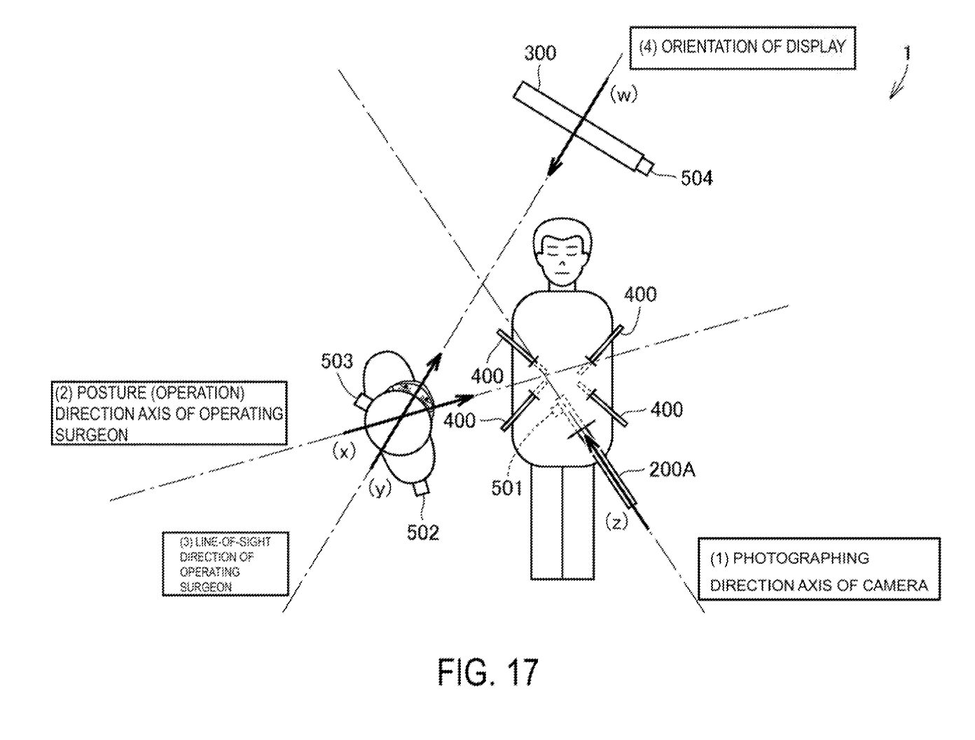

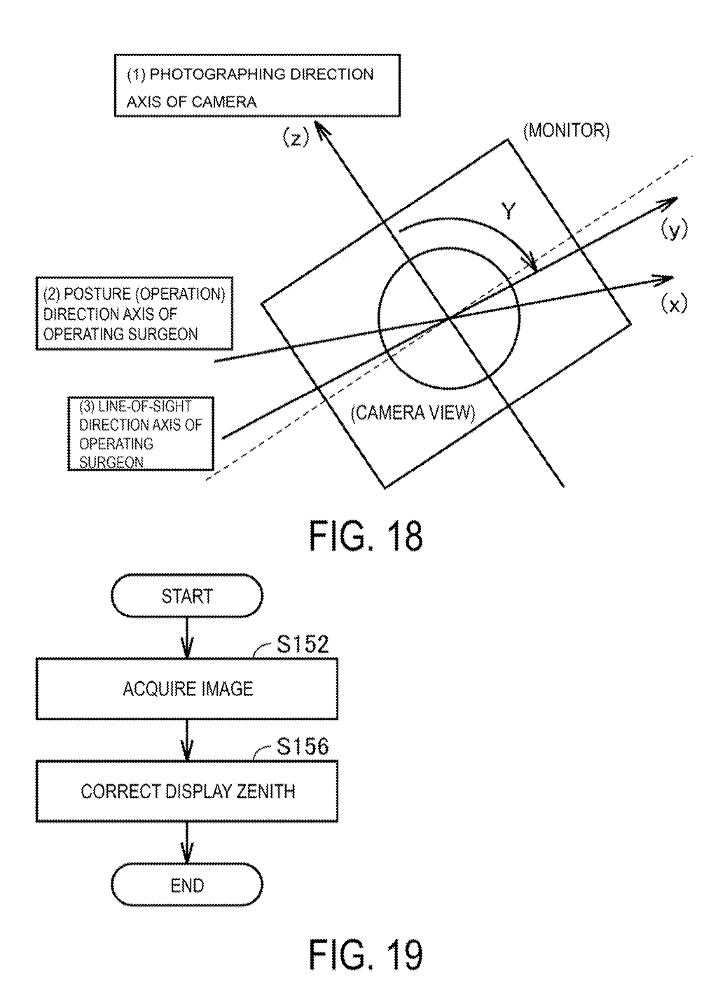

A first information process in a control device 100 according to the present embodiment will be described. FIG. 16 is a flowchart illustrating the first information process in the control device 100 according to the present embodiment. FIG. 17 is a plan view illustrating a relationship in orientation and a positional relationship among the camera 200A after the image photographing direction of the camera 200A is changed in accordance with the orientation of the subject, according to the present embodiment, the body of an operating surgeon, the head of the operating surgeon, and a display 300. FIG. 18 is an image diagram illustrating a relationship among the image photographing direction of the camera 200A after the image photographing direction of the camera 200A is changed in accordance with the orientation of the subject, according to the present embodiment, an orientation of the body of the operating surgeon, a line-of-sight direction of the operating surgeon, and the orientation of the display 300.

Referring to FIGS. 16 to 18, a CPU 110 of the control device 100 periodically acquires, from a first sensor unit 501 through a communication interface 160, vector data indicating an image photographing direction (z) of the camera 200A (step S102).

The CPU 110 acquires, from any one of the sensor units 501 to 505 through the communication interface 160, vector data indicating the orientation of the subject (step S504).

The CPU 110 changes the image photographing direction of the camera 200A based on the orientation of the subject (step S506). To be more specific, the CPU 110 transmits, to the camera 200A through the communication interface 160, a command to control a sensor horizontal direction changing unit 220 and a sensor vertical direction changing unit 230 of the camera 200A, and thus the image photographing direction of the camera 200A is made to turn toward the orientation of the subject “as much as possible”. Then, the CPU 110 transmits, to the camera 200A through the communication interface 160, a command to control a sensor rotation unit 240 of the camera 200A, and thus the image sensor 210 at the tip end of the camera 200A is rotated taking the image photographing direction as an axis so that the upper side of the photographed image comes closest to the actual vertical upper side.

The CPU 110 acquires an angle of the orientation of the subject corresponding to an orientation (z) of the camera on a plane after the change of direction, as a display correction angle Y by which the image is to be rotated and corrected (step S508). In a case where the subject is in the line-of-sight direction of the operating surgeon, the CPU 110 calculates the display correction angle Y based on an equation given below.

Y=y−z (1)

Next, a second information process in the control device 100 according to the present embodiment will be described. FIG. 19 is a flowchart illustrating the second information process in the control device 100 according to the present embodiment.

Referring to FIG. 19, the CPU 110 of the control device 100 acquires image data from the camera 200A through the communication interface 160 (step S152).

The CPU 110 further rotates the image lay −Y degrees based on the display correction angle Y (step S156). The CPU 110 causes the rotated image to be display the display 300 through the communication interface 160.

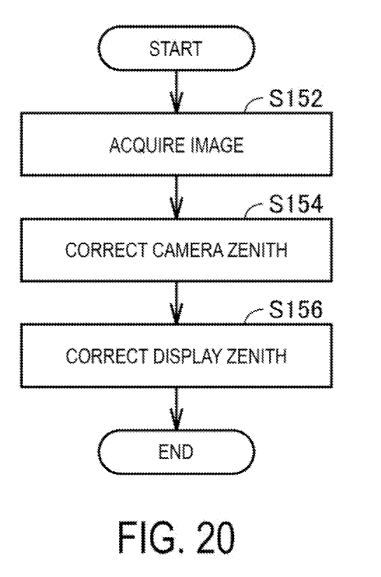

In a case where the camera 200A cannot be rotated taking the image photographing direction as an axis so that the upper side of the photographed image becomes closest to the actual vertical upper side, as illustrated in FIG. 20, the CPU 110 may rotate the image to make an upper direction of the photographed image be closest to an actual vertical upper direction, based on the posture of the camera 200A acquired from the first sensor unit 501, that is, a slant angle of the photographed image screen as a step S154, following the step S152.

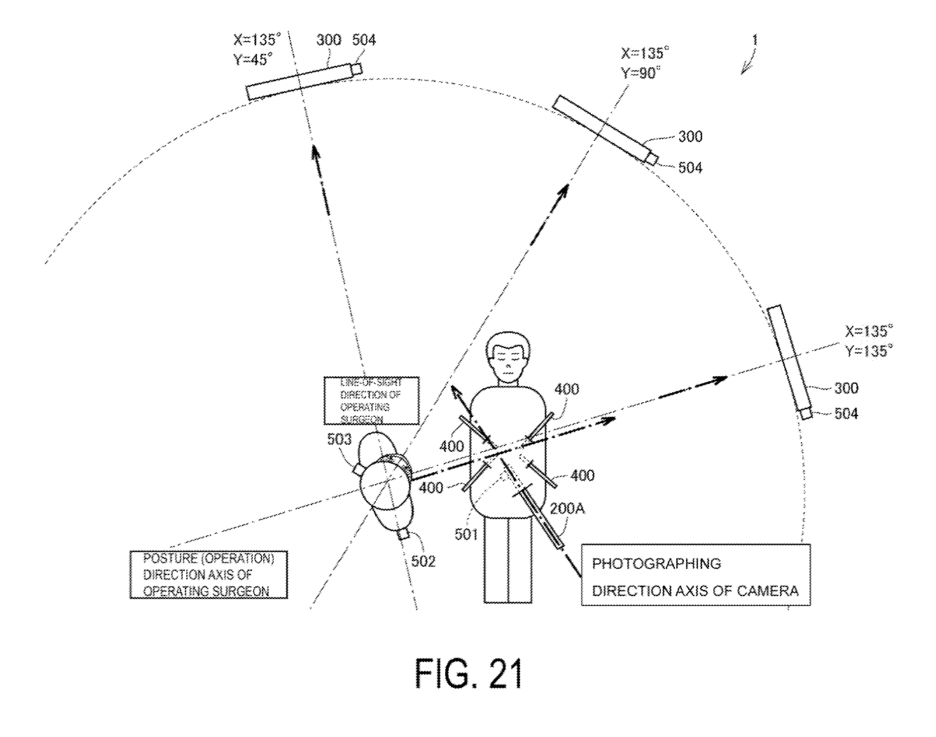

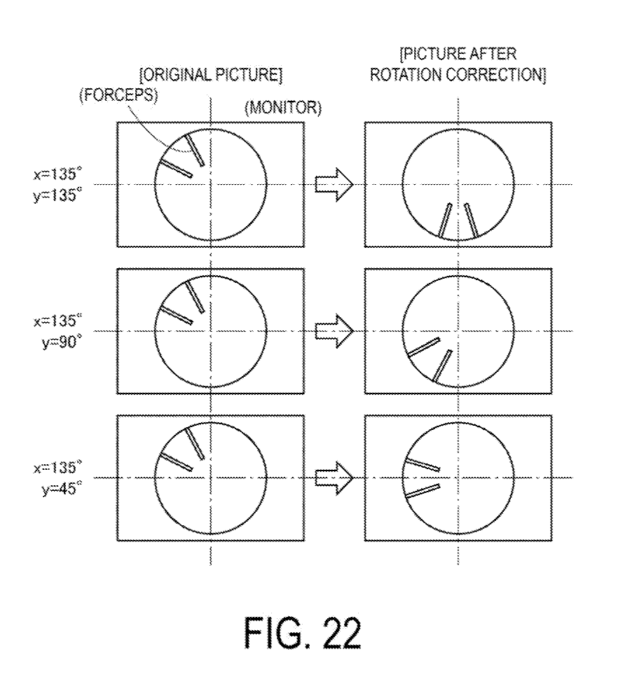

FIG. 21 is an image diagram illustrating actual operations of an image output system 1 according to the present embodiment. FIG. 22 is an image diagram illustrating actual operation results of the display 300 of the image output system 1 according to the present embodiment.

Referring to FIGS. 21 and 22, in the present embodiment, the image photographing direction of the camera 200A is turned toward the orientation of the subject “as much as possible”. Then, the image is further rotated and displayed based on the orientation of the subject and the image photographing direction of the camera 200 after the direction is changed, and thus the rotation angle of the image to be displayed is adjusted as a direction of the operating surgeon toward the display 300 changes. With this, it is possible for the operating surgeon to recognize the positional relationship among the constituent elements with ease as compared with the prior art, and as a result the operation can be easily performed.

More specifically, on the screen of the display 300 in FIG. 22, among four treatment instruments 400 in FIG. 21, two treatment instruments 400 prepared on the operating surgeon side are displayed.

In a case where the orientation of the camera 200 in FIG. 22 is taken as 0 degrees, when an orientation x of the body of the operating surgeon is 135 degrees and an orientation y of the line-of-sight of the operating surgeon is 135 degrees, a correction angle Y, which is obtained by an expression of (Y=y−z), becomes equal to 135 degrees. Therefore, the CPU 110 displays a screen on the display 300 in which the image is rotated by 135 degrees in the counterclockwise direction.

In the case where the orientation of the camera 200 in FIG. 22 is taken as 0 degrees, when the orientation x of the body of the operating surgeon is 135 degrees and the orientation y of the line-of-sight of the operating surgeon is 90 degrees, the correction angle Y, which is obtained by the expression of (Y=y−z), becomes equal to 90 degrees. Therefore, the CPU 110 displays a screen on the display 300 in which the image is rotated by 90 degrees in the counterclockwise direction.

In the case where the orientation of the camera 200 in FIG. 22 is taken as 0 degrees, when the orientation x of the body of the operating surgeon is 135 degrees and the orientation y of the line-of-sight of the operating surgeon is 45 degrees, the correction angle Y, which is obtained by the expression of (Y=y−z), becomes equal to 45 degrees. Therefore, the CPU 110 displays a screen on the display 300 in which the image is rotated by 45 degrees in the counterclockwise direction. As discussed above, since the photographed image is rotated and displayed based on a prescribed rule, the operating surgeon is able to easily understand the positional relationship among the organs, the treatment instruments 400, and the like.

It is preferable that a user such as an operating surgeon be able to fine-tune the rotation angle of the image through the operation unit 140 of the control device 100.

In addition, information of the image rotation may be updated in real time. The frequency of the information updates may be adjustable 60 seconds, 10 seconds, one second, or a timing determined by the operating surgeon by manual operation). Alternatively, a configuration may be selected in which the positional relationship among the devices, the rotation angle of the image, and the like are “confirmed” before the operation, and the rotation angle of the image is not changed during the operation.

### Sixth Embodiment

Further, in the present embodiment, a control device 100 accepts and registers designation of factors, as base data for targets of an image photographing direction for a camera 200A, for each user such as an operating surgeon, a surgical assistant, or the like.

For example, a user accepts, through an operation unit 140 of the control device 100, designation of factors as the base data for the targets of the image photographing direction for the camera 200A, for example, a line-of-sight direction of the operating surgeon, an orientation of the body of the operating surgeon, an orientation of a treatment instrument 400, an orientation of a display 300, and the like. A CPU 110 of the control device 100 stores, in a memory 120, a correspondence relationship between information for identifying the user and information for identifying the factors as the base data for the targets of the image photographing direction for the camera 200A. Note that the data of the correspondence relationship may be stored in another device that can be accessed by the control device 100.

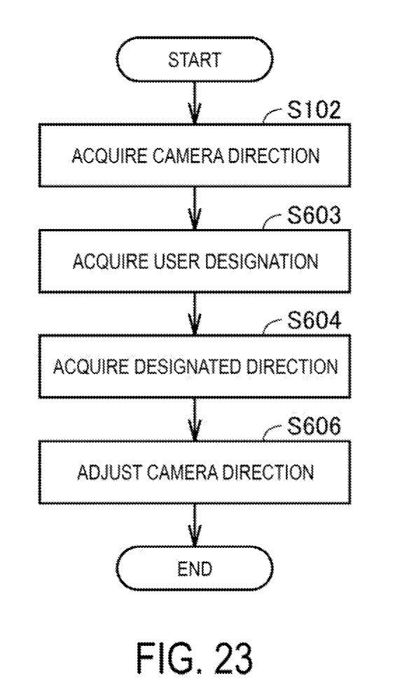

FIG. 23 is a flowchart illustrating a first information process in the control device 100 according to the present embodiment. Note that the CPU 110 of the control device 100 accepts a user ID and the like beforehand through the operation unit 140.

Referring to FIG. 23, the CPU 110 of the control device 100 periodically acquires, from a first sensor unit 501 through a communication interface 160, vector data indicating an image photographing direction (z) of the camera 200A (step S102).

The CPU 110 identifies the factors as the base data for the targets of image photographing direction for the camera 200A based on the information for identifying the current user using the system at the time (step S603).

The CPU 110 acquires, from any one of the sensor units 501 to 505 through the communication interface 160, vector data indicating an orientation of a designated subject (step S604).

The CPU 110 changes the image photographing direction of the camera 200A based on the orientation of the subject (step S606). To be more specific, the CPU 110 transmits, to the camera 200A through the communication interface 160, a command to control a sensor horizontal direction changing unit 220 and a sensor vertical direction changing unit 230 of the camera 200A, and thus the image photographing direction of the camera 200A is made to turn toward the orientation of the subject “as much as possible”. Then, the CPU 110 transmits, to the camera 200A through the communication interface 160, a command to control a sensor rotation unit 240 of the camera 200A, and thus the image sensor 210 at the tip end of the camera 200A is rotated taking the image photographing direction as an axis so that the upper side of the photographed image comes closest to the actual vertical upper side.

Furthermore, as described in the fifth embodiment, the CPU 110 may further acquire an angle of the orientation of the subject corresponding to the orientation (z) of the camera after the change of orientation on a plane, as a display correction angle Y by which the image is to be rotated and corrected (step S508 in FIG. 16).

### Seventh Embodiment

It is only required for the factors to be capable of making adjustments in such a manner that the photographed image can be easily seen by an operating surgeon as a medical specialist, a positional relationship among the organs being displayed, treatment instruments 400 and the like can be easily recognized by the operating surgeon, or the operation can be performed with ease by the operating surgeon, and the present disclosure is not limited to the embodiments such as the first to sixth embodiments in which the image photographing direction of the camera is adjusted based on the factors such as the line-of-sight direction of the operating surgeon, the orientation of the body of the operating surgeon, the orientation of the treatment instrument 400, and the orientation of the display 300 and the image photographing direction of the camera may be adjusted based on the plurality of factors among the above-mentioned factors.

It is possible to study and derive a more optimal relationship in position (rotation angle) by accumulating the data acquired from the sensors and the like. Extracting the characteristics (habits) of a practitioner of surgery as data may contribute to the improvement of skills of the practitioner of surgery.

### Other Application Examples

It is needless to say that an aspect of the present disclosure can be applied also in a case where the stated aspect is achieved by supplying a program to a system or a device. Further, it is also possible to obtain an effect of an aspect of the present disclosure by a scheme in which a storage medium (or a memory) storing a program expressed by software to achieve the stated aspect of the present disclosure is supplied to a system or a device, and a computer (such as a CPU or MPU) of the system or the device reads out the program code stored in the storage medium and then performs the program code.

In this case, the program code itself having been read out from the storage medium enables the functions of the above embodiments, and thus the storage medium storing the program code constitutes an aspect of the present disclosure.

Further, needless to say, an aspect of the present disclosure includes not only the case in which the computer performs the program code having been read out to enable the functions of the above-described embodiments, but also a case in which an operating system (OS) or the like working on the computer performs part or all of actual processes in accordance with commands of the program code so that the functions of the above-described embodiments are enabled by the stated processes.

Also needless to say, an aspect of the present disclosure includes a case in which, after the program code having been read out from the storage medium is written into another storage medium provided in a function enhancement board inserted in the computer, a function enhancement unit connected to the computer or the like, a CPU or the like provided in the function enhancement hoard, the function enhancement unit or the like performs part or all of the actual processes in accordance with the commands of the program code so that the functions of the above embodiments are enabled by the stated processes.

In addition, in the image output system and the control device described in the aforementioned embodiments, each of the blocks thereof may be individually formed in a single chip by a semiconductor device such as an LSI, or may be formed in a single chip in such a manner as to include part or all of the block.

Although the term LSI is used here, an LSI is also called an IC, system LSI, super LSI, or ultra LSI in some case depending on the degree of integration.

The method of circuit integration is not limited to an LSI, and the circuit integration may be achieved by using a dedicated circuit or a general-purpose processor. After the manufacture of an LSI, a Field Programmable Gate Array (FPGA) that can be programmed, a reconfigurable processor in which connections, configurations or the like of circuit cells inside the LSI can be reconfigured, or the like may be used.

Further, in a case where a technology of circuit integration capable of replacing LSIs becomes available by the progress of the semiconductor technology or another technology derived therefrom, it is needless to say that the functional blocks may be integrated by using the stated technology. It may be possible to apply biotechnology or the like in this field.

The processes of the aforementioned embodiments may be implemented by hardware or software (including a case where the processes are implemented along with an operating system (OS), middleware, or a prescribed library). In addition, the above processes may be implemented by a mixed process of software and hardware. In the case where the image output system and the control device according to the above-described embodiments are implemented by hardware, it is needless to say that the timing for carrying out each of the processes need to be adjusted. In the above-described embodiments, detailed description of the timing adjustments of various signals necessary to be considered in actual design is omitted for the sake of convenience in explanation.

### Supplement

In the above-described first to seventh embodiments, the image photographing system 1 including the camera 200A, and the control device 100 configured to communicate with the camera 200A is provided. The control device 100 adjusts the image photographing direction of the camera 200A based on an orientation of the camera 200A and an orientation of the prescribed subject.

It is preferable for the image photographing system 1 to further include a display 300. The control device 100 causes an image photographed by the camera 200A to be rotated based on an angle between an orientation of the camera 200A and an orientation of the prescribed subject on a plane, and then causes the photographed image to be outputted to the display 300.

It is preferable for the control device 100 to accept the designation of the prescribed subject for each user.

In the above-described embodiments, provided is an image photographing method including a step of acquiring an orientation of the camera 200A, a step of acquiring an orientation of the prescribed subject and a step of adjusting an image photographing direction of the camera 200A based on the orientation of the camera 200A and the orientation of the prescribed subject.

In the above embodiments, provided is the control device 100 including the communication interface 160 configured to communicate with the camera 200A, and the processor 110. The processor 110 adjusts the image photographing direction of the camera 200A based on the orientation of the camera 200A and the orientation of the prescribed subject through the communication interface 160.

The embodiments disclosed herein are to be understood as being in all ways exemplary and in no way limiting. The scope of the present disclosure is defined not by the foregoing descriptions but by the appended claims, and is intended to include all changes equivalent in meaning and scope to the appended claims.

### REFERENCE SIGNS LIST

1 image photographing system (Picture output system
100 Control device
110 Processor (CPU)
120 Memory
140 Operation unit
160 Communication interface
200A Camera
200B Camera controller
210 Image sensor
220 Sensor horizontal direction changing unit
230 Sensor vertical direction changing unit
240 Sensor rotation unit
260 Communication interface
300 Display
400 Treatment instrument
501 to 505 Sensor unit

1. An image photographing system comprising:
a camera; and
a control device configured to communicate with the camera,
wherein the control device is configured to adjust an image photographing direction of the camera based on an orientation of the camera and an orientation of a prescribed subject.

2. The image photographing system according to claim 1, further comprising a display,
wherein the control device outputs an image obtained by rotating an image photographed by the camera based on an orientation of the camera to display.

3. The image photographing system according to claim 1,
wherein the control device accepts designation of the prescribed subject for each user.

4. An image photographing method comprising:
a step of acquiring an orientation of a camera;
a step of acquiring an orientation of a prescribed subject; and
a step of adjusting the image photographing direction of the camera based on an orientation of the camera and an orientation of the prescribed subject.

5. A control device comprising:
a communication interface configured to communicate with a camera; and
a processor,
wherein the processor adjusts an image photographing direction of the camera based on an orientation of the camera and an orientation of the prescribed subject through the communication interface.
